(19) United States
(12) Patent Application Publication
(10) Pub. No.: US 2025/0186141 A1
(43) Pub. Date: Jun. 12, 2025

(54) INSTRUMENTS FOR SURGICAL ROBOTIC SYSTEM AND INTERFACES FOR THE SAME

(71) Applicant: Distalmotion SA, Epalinges (CH)

(72) Inventors: Lancelot DUPONT, Pully (CH); Julien CHASSOT, Lechelles (CH)

(21) Appl. No.: 18/811,640

(22) Filed: Aug. 21, 2024

Related U.S. Application Data
(63) Continuation of application No. 18/603,045, filed on Mar. 12, 2024, now Pat. No. 12,114,945.
Application No. 18/603,045 is a continuation of application No. PCT/IB2022/058437, filed on Sep. 8, 2022.
(60) Provisional application No. 63/243,527, filed on Sep. 13, 2021.

(51) Int. Cl. A61B 34/30; A61B 17/00; A61B 17/32; A61B 34/35

(52) U.S. Cl. CPC A61B 34/30; A61B 17/320092; A61B 34/35; A61B 2017/00367; A61B 2017/320093; A61B 2034/305

(57) ABSTRACT

Systems and methods for actuating an end-effector of a surgical instrument are provided. The surgical instrument includes a surgical instrument interface operatively coupled to an instrument shaft having an end-effector. The surgical instrument interface includes a pair of actuators slidably disposed within longitudinal openings of a housing of the surgical instrument interface and engaged with a grooved opening of a barrel rotatably disposed within the housing, such that translational movement of the actuators within the longitudinal openings is converted to rotational movement of the barrel along the grooved opening. The surgical instrument interface further may include a torsion spring pre-loaded with a predetermined torque such that rotation of the barrel causes rotation of the instrument shaft if the torque generated between the barrel and the instrument shaft is less than the predetermined torque of the spring. Rotation of the instrument shaft actuates the end-effector via a cam mechanism.

This application claims priority to U.S. Provisional Patent Application Ser. No. 63/243,527, filed Sep. 13, 2021, the entire contents of which are incorporated herein by reference.

### FIELD OF USE

The present disclosure is directed to surgical instruments and interfaces for the same such as, for example, removable surgical instruments for remotely actuated surgical robot systems.

### BACKGROUND

Numerous environments and applications call for remote actuation with teleoperated surgical devices. These applications include the ability to perform fine manipulation, to manipulate in confined spaces, manipulate in dangerous or contaminated environments, in clean-room or sterile environments and in surgical environments, whether open field or minimally invasive. While these applications vary, along with parameters such as precise tolerances and the level of skill of the end user, each demands many of the same features from a teleoperated system, such as the ability to carry out dexterous manipulation with high precision.

Surgical applications are discussed in the following disclosure in more detail as exemplary of applications for a teleoperated device system where known devices exist but significant shortcomings are evident in previously-known systems and methods.

Open surgery is still the preferred method for many surgical procedures. It has been used by the medical community for many decades and typically required making long incisions in the abdomen or other area of the body, through which traditional surgical tools are inserted. Due to such incisions, this extremely invasive approach results in substantial blood loss during surgery and, typically, long and painful recuperation periods in a hospital setting.

Laparoscopy, a minimally invasive technique, was developed to overcome some of the disadvantages of open surgery. Instead of large through-wall incisions, several small openings are made in the patient through which long and thin surgical instruments and endoscopic cameras are inserted. The minimally invasive nature of laparoscopic procedures reduces blood loss and pain and shortens hospital stays. When performed by experienced surgeons, a laparoscopic technique can attain clinical outcomes similar to open surgery. However, despite the above-mentioned advantages, laparoscopy requires a high degree of skill to successfully manipulate the rigid and long instrumentation used in such procedures. Typically, the entry incision acts as a point of rotation, decreasing the freedom for positioning and orientating the instruments inside the patient. The movements of the surgeon's hand about this incision point are inverted and scaled-up relative to the instrument tip (“fulcrum effect”), which reduces dexterity and sensitivity and magnifies any tremors of the surgeon's hands. In addition, the long and straight instruments force the surgeon to work in an uncomfortable posture for hands, arms and body, which can be tremendously tiring during a prolonged procedure. Therefore, due to these drawbacks of laparoscopic instrumentation, minimally invasive techniques are mainly limited to use in simple surgeries, while only a small minority of surgeons is able to use such instrumentation and methods in complex procedures.

To overcome the foregoing limitations of previously-known systems, surgical robotic systems were developed to provide an easier-to-use approach to complex minimally invasive surgeries. By means of a computerized robotic interface, those systems enable the performance of remote laparoscopy where the surgeon sits at a console manipulating two master manipulators to perform the operation through several small incisions. Like laparoscopy, the robotic approach is also minimally invasive, providing the above-mentioned advantages over open surgery with respect to reduced pain, blood loss, and recuperation time. In addition, it also offers better ergonomy for the surgeon compared to open and laparoscopic techniques, improved dexterity, precision, and tremor suppression, and the removal of the fulcrum effect. Although being technically easier, robotic surgery still involves several drawbacks. One major disadvantage of previously-known robotic surgical systems relates to the extremely high complexity of such systems, which contain four to five robotic arms to replace the hands of both the surgeon and the assistant, integrated endoscopic imaging systems, as well as the ability to perform remote surgery, leading to huge capital costs for acquisition and maintenance, and limiting the affordably for the majority of surgical departments worldwide. Another drawback of these systems is the bulkiness of previously-known surgical robots, which compete for precious space within the operating room environment and significantly increasing preparation time. Access to the patient thus may be impaired, which raises safety concerns.

For example, the da Vinci® surgical systems (available by Intuitive Surgical, Inc., Sunnyvale, California, USA) is a robotic surgical system for allowing performance of remote laparoscopy by a surgeon. However, the da Vinci® surgical systems are very complex robotic systems, with each system costing around $2,000,000 per robot, $150,000 per year for servicing, and $2,000 per surgery for surgical instruments. The da Vinci® surgical system also requires a lot of space in the operating room, making it hard to move around to a desired location within the operating room, and difficult to switch between forward and reverse surgical workspaces (also known as multi-quadrant surgery).

Moreover, as the surgeon's operating console is typically positioned away from the surgical site, the surgeon and the operating console are not in the sterile zone of the operating room. If the surgeon's operating console is not sterile, the surgeon is not permitted to attend to the patient if necessary without undergoing additional sterilization procedures. During certain surgical operations, a surgeon may need to intervene at a moment's notice, and current bulky robotic systems may prevent the surgeon from quickly accessing the surgical site on the patient in a timely, life-saving manner.

WO97/43942 to Madhani, WO98/25666 to Cooper, and U.S. Patent Application Publication No. 2010/0011900 to Burbank each discloses a robotic teleoperated surgical instrument designed to replicate a surgeon's hand movements inside the patient's body. By means of a computerized, robotic interface, the instrument enables the performance of remote laparoscopy, in which the surgeon, seated at a console and manipulating two joysticks, performs the operation through several small incisions. Those systems do not have autonomy or artificial intelligence, being essentially a sophisticated tool that is fully controlled by the surgeon. The control commands are transmitted between the robotic master and robotic slave by a complex computer-controlled mechatronic system, which is extremely costly to produce and maintain and requires considerable training for the hospital staff.

WO2013/014621 to Beira, the entire contents of which are incorporated herein by reference, describes a mechanical teleoperated device for remote manipulation which comprises master-slave configuration including a slave unit driven by a kinematically equivalent master unit, such that each part of the slave unit mimics the movement of a corresponding part of the master unit. A typical master-slave telemanipulator provides movement in seven degrees-of-freedom. Specifically, these degrees of freedom include three translational macro movements, e.g., inward/outward, upward/downward, and left/right degrees-of-freedoms, and four micro movements including one rotational degree-of-freedom, e.g., roll, two articulation degrees-of-freedom, e.g., yaw and pitch, and one actuation degree-of-freedom, e.g., open/close. Although the mechanical transmission system described in that publication is well adapted to the device, the low-friction routing of the cables from handles through the entire kinematic chain to the instruments is costly, complex, bulky, and requires precise calibration and careful handling and maintenance.

In addition, previously-known purely mechanical solutions do not offer wrist alignment, low device complexity, low mass and inertia, high surgical volume, and good haptic feedback. For example, with a purely mechanical teleoperated device, in order to perform a pure pronosupination/roll movement of the instrument, the surgeon typically has to perform a combined pronosupination/roll movement of his hand/forearm as well as a translational movement on a curved path with his wrist. Such movements are complex to execute properly, and if not done properly, the end-effector pitches and yaws creating undesired parasitic movements.

Further, the routing of the articulation and actuation degrees-of-freedom cables through mechanical telemanipulators may limit the dexterity of the angular range of the various joints of the telemanipulator link-and-joint structure. This in turn limits the available surgical volume of the instruments accessible within the patient. During rapid movements of the mechanical telemanipulators, inertia of the telemanipulators also may be disturbing and result in over-shoot of the target and fatigue of the surgeon's hand. Part of this mass can be attributed to parts and components required to route the actuation and articulation degrees-of-freedom.

Accordingly, it would be desirable to provide remotely actuated surgical robot systems having robotic telemanipulators that are well adapted for use by the surgeon, seamlessly integrated into the operation room, allow for a surgeon to work between the robot and the patient in a sterile manner, are relatively low cost, and/or permit integrated laparoscopy.

It would further be desirable to provide a remotely actuated surgical robot having mechanical and/or electromechanical telemanipulators.

Moreover, it is desirable to provide advanced energy instrumentation on a robotic system, such as ultrasonic dissectors. Accordingly, there is a need for surgical instrumentation having a rigid and plain sonotrode coaxial with the instrument shaft, which does not allow for the use of traditional cable driven transmission. Thus, it would further be desirable to provide surgical instruments having a surgical instrument interface that does not require cables to actuate the end-effector of the surgical instrument. In addition, as it is desirable for the closing force of such instruments to be precisely controlled, there is a need for such surgical instruments to have reduced torque.

### SUMMARY

The present invention overcomes the drawbacks of previously-known systems by providing surgical instruments having a surgical instrument interface for interchangeably coupling with a patient hub of a patient console of a robotic telemanipulator system and/or for a handheld surgical system, and for actuating an end-effector of the surgical instrument without cable transmission.

As will be understood by a person having ordinary skill in the art, the term “master” used herein refers to components controlled by the surgeon and may be referred to as “surgeon,” and the term “slave” used herein refers to components that interact with the patient undergoing the surgery and may be referred to as “patient.” For example, the terms “master console” and “surgeon console” are interchangeable and the terms “slave console” and “patient console” are interchangeable, etc.

In accordance with one aspect of the present disclosure, a surgical instrument interface operatively coupled to an instrument shaft having an end-effector is provided. The surgical instrument interface includes a housing having a longitudinal opening, and a barrel rotatably disposed within the housing. The barrel includes a grooved opening extending circumferentially along at least a portion of the barrel. Moreover, the surgical instrument interface further includes an actuator slidably disposed within the longitudinal opening and engaged with the grooved opening, such that translational movement of the actuator along the longitudinal opening causes the barrel to rotate along the grooved opening to thereby rotate the instrument shaft. In some embodiments, the surgical instrument interface may include a spring having a proximal end coupled to the barrel and a distal end coupled to the instrument shaft. The spring may be pre-loaded with a predetermined torque. Accordingly, translational movement of the actuator along the longitudinal opening may cause the barrel to rotate along the grooved opening to thereby rotate the instrument shaft via the spring.

For example, rotation of the barrel causes the instrument shaft to rotate when a torque generated by rotation of the barrel between the barrel and the instrument shaft is less than the predetermined torque of the spring. In addition, when the torque generated by rotation of the barrel between the barrel and the instrument shaft is less than the predetermined torque of the spring, the spring transmits the torque to the instrument shaft to rotate the instrument shaft in a first direction.

In some embodiments, the barrel includes a cam stop that engages with a cam ring fixed to the instrument shaft. Thus, when the torque generated by rotation of the barrel between the barrel and the instrument shaft is less than the predetermined torque of the spring, the cam stop transmits the torque to the instrument shaft via the cam ring to rotate the instrument shaft in a second direction opposite to the first direction. For example, the barrel stop extends proximally from a proximal end of the barrel, and the cam ring has a distal opening sized and shaped to receive the cam stop and to permit the cam stop to circumferentially move within the distal opening.

Moreover, when a torque generated by rotation of the barrel between the barrel and the instrument shaft is greater than the predetermined torque of the spring, rotation of the barrel in a first direction causes the spring to compress torsionally such that rotation torque of the barrel in the first direction limits the torque transmitted to the shaft to the torsion torque of the spring. Additionally, rotation of the barrel in a second direction opposite to the first direction causes the spring to absorb movement of the barrel until the barrel is able to transmit torque to the instrument shaft. In some embodiments, the distal end of the spring is coupled to the instrument shaft via a fixation ring, which may be actuated to pre-load the spring with the predetermined torque.

The housing may include a second longitudinal opening, such that the surgical instrument interface further includes a second actuator slidably disposed within the second longitudinal opening and engaged with the grooved opening. Accordingly, translational movement of the second actuator along the second longitudinal opening causes the barrel to rotate along the grooved opening to thereby rotate the instrument shaft, e.g., via the spring. The grooved opening may have a shape that permits the first and second actuators to move in opposite directions along the respective longitudinal openings to thereby rotate the barrel. In some embodiments, the grooved opening includes first and second grooved openings, each of the first and second grooved openings sized and shaped to be engaged with a corresponding actuator. In some embodiments, the end-effector may emit ultrasonic energy. Moreover, the end-effector may include a pair of jaws. Accordingly, rotation of the instrument shaft may cause a first jaw of the pair of jaws to move relative to a second jaw of the pair of jaws of the end-effector to thereby cause the end-effector to open and close. Preferably, the first jaw is operatively coupled to the instrument shaft via a cam mechanism such that rotation of the instrument shaft causes the end-effector to open and close.

In accordance with another aspect of the present disclosure, a surgical instrument that may be attached to a surgical platform is provided. The surgical instrument includes the surgical instrument interface described above, an instrument shaft operatively coupled to the surgical instrument interface, and an end-effector disposed at a distal end of the instrument shaft.

In accordance with yet another aspect of the present disclosure, a surgical robot system that may be releasably coupled to the surgical instrument described above is provided. The surgical robot system may be a surgical platform having a patient console, which may releasably engage with the actuator of the surgical instrument interface.

In accordance with another aspect of the present disclosure, a method for actuating an end-effector of a surgical instrument is provided. The method includes selecting a surgical instrument interface operatively coupled to an instrument shaft having the end-effector, the surgical instrument interface including a housing having a longitudinal opening, a barrel rotatably disposed within the housing; and translationally moving an actuator disposed within the longitudinal opening and engaged with a grooved opening extending circumferentially along at least a portion of the barrel, to thereby cause the barrel to rotate along the grooved opening which causes rotation of the instrument shaft. In some embodiments, the method includes selecting a surgical instrument interface further having a spring coupled to the barrel and the instrument shaft, such that the spring may be pre-loaded with a predetermined torque. Accordingly, translationally moving the actuator causes the barrel to rotate along the grooved opening which causes rotation of the instrument shaft via the spring when a torque generated by rotation of the barrel between the barrel and the instrument shaft is less than the predetermined torque of the spring. When the torque generated by rotation of the barrel between the barrel and the instrument shaft is greater than the predetermined torque of the spring, rotation of the barrel causes the spring to compress torsionally, such that rotation torque of the barrel limits the torque transmitted to the shaft to the torsion torque of the spring.

The method further includes translationally moving a second actuator disposed within a second longitudinal opening of the housing and engaged with a second grooved opening extending circumferentially along at least a portion of the barrel, to thereby cause the barrel to rotate along the second grooved opening which causes rotation of the instrument shaft, e.g., via the spring when the torque generated by rotation of the barrel between the barrel and the instrument shaft is less than the predetermined torque of the spring. The actuators translationally move in opposite directions. Accordingly, rotation of the instrument shaft may cause a first jaw of the end-effector to move relative to a second jaw of the end-effector via a cam mechanism to thereby cause the end-effector to open and close. The method further may include emitting ultrasonic energy via the end-effector.

### BRIEF DESCRIPTION OF THE DRAWINGS

FIG. 1 illustrates an exemplary remotely actuated surgical robot system having robotic telemanipulators constructed in accordance with the principles of the present disclosure.

FIG. 2 illustrates an exemplary patient console of the system of FIG. 1 constructed in accordance with the principles of the present disclosure.

FIG. 3A illustrates an exemplary patient hub of the patient console of FIG. 2 constructed in accordance with the principles of the present disclosure.

FIG. 3B illustrates an exemplary sterile shield coupled to the patient hub of FIG. 3A in accordance with the principles of the present disclosure.

FIGS. 4A and 4B illustrate an exemplary surgical instrument constructed in accordance with the principles of the present disclosure.

FIG. 5 illustrates an exemplary rotation barrel of the surgical instrument of FIGS. 4A and 4B constructed in accordance with the principles of the present disclosure.

FIGS. 6A and 6B illustrate exemplary actuators of the surgical instrument of FIGS. 4A and 4B in accordance with the principles of the present disclosure.

FIGS. 7A and 7B illustrate actuation of the surgical instrument of FIGS. 4A and 4B when the torque generated is less than the spring torque in accordance with the principles of the present disclosure.

FIGS. 8A and 8B illustrate actuation of the surgical instrument of FIGS. 4A and 4B when the torque generated is greater than the spring torque in accordance with the principles of the present disclosure.

FIG. 9 is a graph illustrating torque transmission to the instrument shaft relative to the force applied to the actuators in accordance with the principles of the present disclosure.

FIGS. 10A and 10B illustrate an exemplary end-effector of the surgical instrument of FIGS. 4A and 4B constructed in accordance with the principles of the present disclosure.

FIG. 10C illustrates an alternative exemplary end-effector of the surgical instrument constructed in accordance with the principles of the present disclosure.

FIGS. 11A and 11B illustrate actuation of the end-effector of FIGS. 10A and 10B in accordance with the principles of the present disclosure.

FIG. 12 illustrates the surgical instrument of FIGS. 4A and 4B coupled to a handheld surgical system in accordance with the principles of the present disclosure.

### DETAILED DESCRIPTION

A surgical instrument having a surgical instrument interface is described herein. The surgical instrument interface may be used for interchangeably coupling with a patient hub of a patient console of a robotic telemanipulator system and/or with a handheld surgical system, and for actuating an end-effector of the surgical instrument without cable transmission. The surgical instrument interface permits actuation of the end-effector of the surgical instrument by translating translational movement of one or more actuators of the surgical instrument interface into rotational movement of an instrument shaft of the surgical instrument. The rotational movement of the instrument may then be translated to actuation of the end-effector in a degree-of-freedom, e.g., open and close, via, for example, a cam mechanism between the instrument shaft and the end-effector, as described in U.S. 2005/0216045 to Young, the entire contents of which are incorporated herein by reference. Accordingly, this configuration does not require cable transmission to actuate the end-effector. In addition, the end-effector may be actuated to emit ultrasonic energy for various surgical procedures.

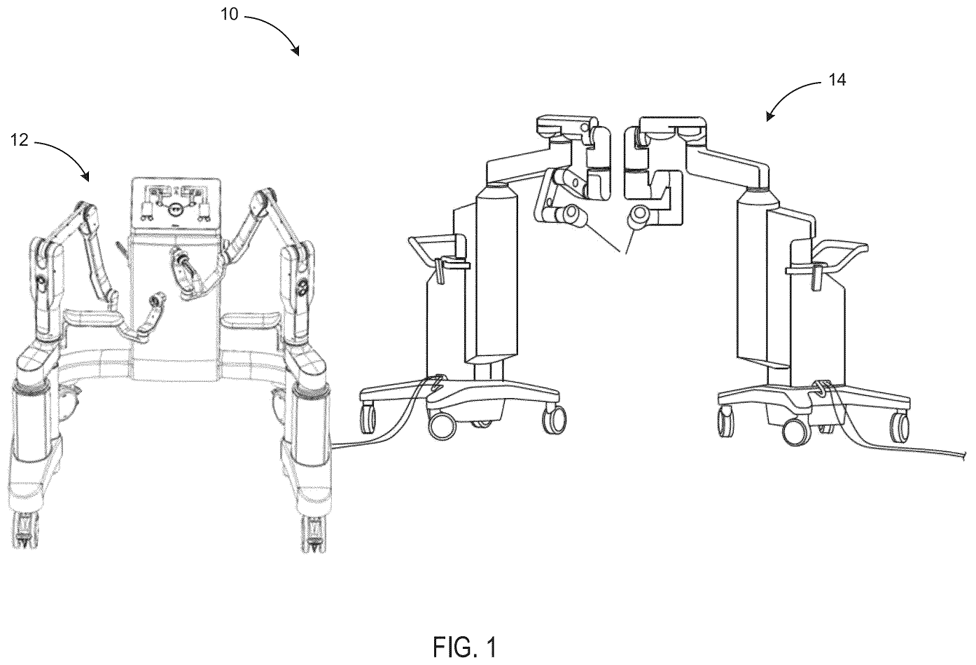

Referring now to FIG. 1, exemplary remotely actuated surgical robot system 10 having robotic telemanipulators is described. Surgical robot system 10 is a surgical platform and may be constructed as described in U.S. Pat. No. 10,413,374 to Chassot and/or International Patent Appl. Pub. No. WO 2020/141487 to Chassot, the entire contents of each of which are incorporated herein by reference. For example, as shown in FIG. 1, surgical robot system 10 includes surgeon console 12 electrically and operatively coupled to patient console 14 via, e.g., electrical cables. Surgeon console 12 includes a plurality of surgeon links interconnected by a plurality of surgeon joints, and patient console 14 includes a plurality of patient links interconnected by a plurality of patient joints.

A plurality of actuators, e.g., preferably motors, coupled to patient console 14 may apply translational macro-movements to an end-effector of patient console 14 responsive to movement applied at surgeon console 12 via a processor-driven control system, e.g., when surgical robot system 10 is in a macro-synchronization state. Additionally, a plurality of actuators, e.g., preferably motors, coupled to patient console 14 may apply micro-movements to an end-effector of patient console 14 responsive to movement applied at a handle of surgeon console 12 via the processor-driven control system, e.g., when surgical robot system 10 is in a micro-synchronization state. Accordingly, the patient links and joints of patient console 14 may move in a manner responsive to movement applied at the handle of surgeon console 12 such that the surgical instrument reproduces the movement applied at the handle of surgeon console 12.

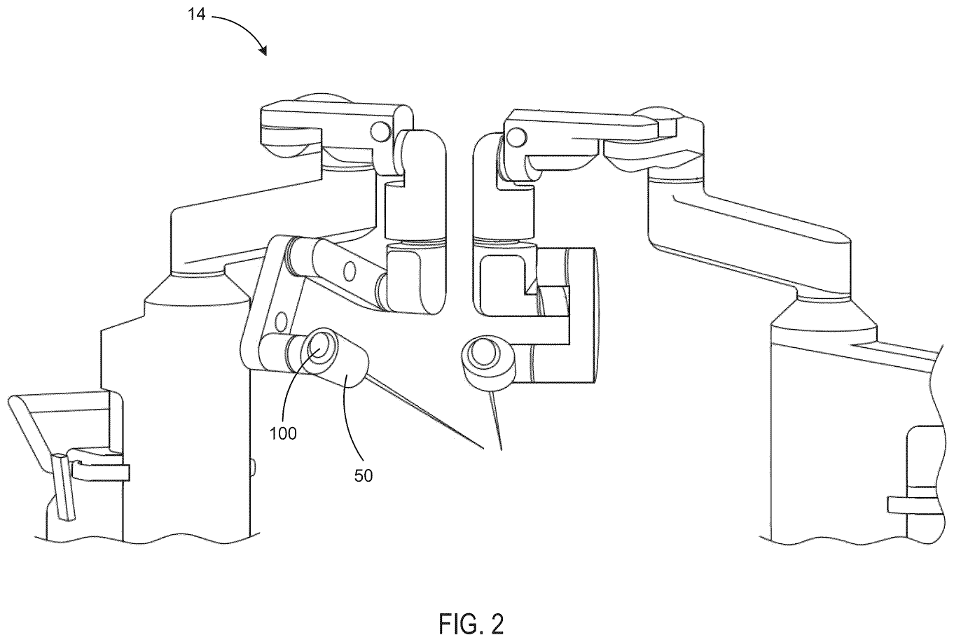

Referring now to FIG. 2, an exemplary patient console of surgical robot system 10 is provided. As shown in FIG. 2, a distal patient link of patient console 14 includes patient hub 50 for releasably engaging with a surgical instrument, e.g., surgical instrument 100, via the surgical instrument interface of the surgical instrument, as described in further detail below. Accordingly, patient console 14 may be actuated to cause surgical instrument 100 to reproduce the movement applied at the handle of surgeon console 12 via patient hub 50.

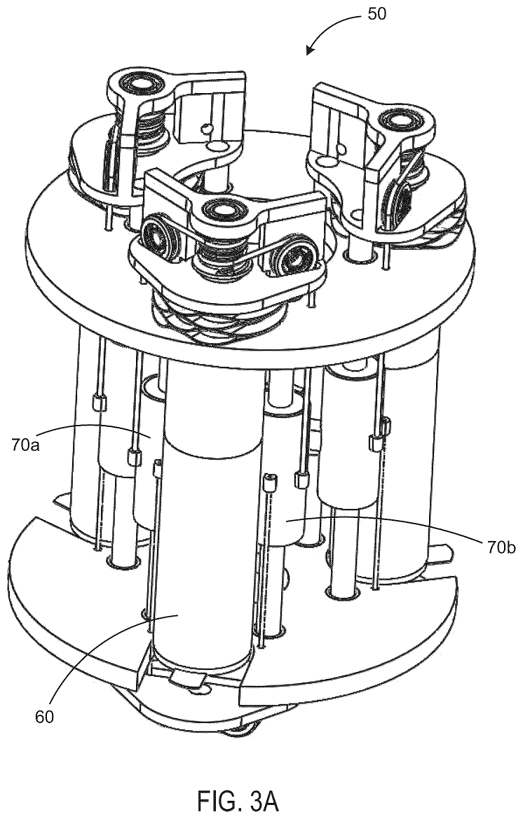

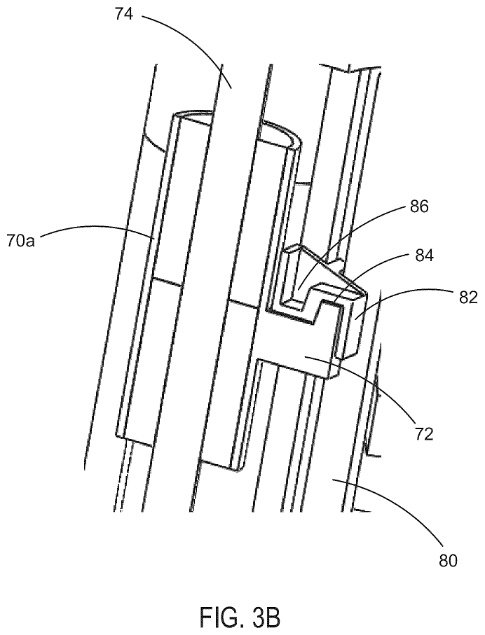

Referring now to FIG. 3A, an exemplary patient hub of patient console 14 is provided. Patient hub 50 includes a translational instrument interface, which may be constructed as described in U.S. Pat. No. 11,058,503 to Chassot, the entire contents of which are incorporated herein by reference. For example, patient hub 50 may include one or more motors 60, each motor configured to actuate a pair of receptacles 70a, 70b. Each receptacle 70a, 70b includes an engager sized and shaped to engage with an actuator of the surgical instrument interface of surgical instrument 100, as described in further detail below. Accordingly, motor 60 may be actuated to cause receptacles 70a, 70b to translationally move in equal and opposite directions, e.g., a system of pulleys and cables or gears, which causes corresponding translational movement of the actuators of the surgical instrument interface of surgical instrument 100. Moreover, patient hub 50 includes a passageway extending therethrough, sized and shaped to receive surgical instrument 100 therethrough, such that the engagers of receptacles 70a, 70b engage with the actuators of the surgical instrument interface of surgical instrument 100.

Referring now to FIG. 3B, an exemplary sterile shield is provided. Sterile shield 80 may be constructed as described in U.S. Pat. No. 11,058,503 to Chassot, and may be positioned between the engagers of patient hub 50, e.g., engager 72 as shown in FIG. 3B, and the actuators of the surgical instrument interface of surgical instrument 100. Sterile shield 80 may include moveable slider 82 to provide a mechanical connection between receptacle 70a of patient hub 50 and the corresponding actuator of surgical instrument 100. As shown in FIG. 3B, sterile shield 80 has first portion 84 having a geometry corresponding to engager 72 such that first portion 84 may engage with engager 72, and second portion 86 having a geometry corresponding to the actuator of surgical instrument 100 such that second portion 86 may engage with the actuator of surgical instrument 100. Sterile shield 80 also may be integrated on a sterile sleeve to create a sterile barrier for the entire patient console 14.

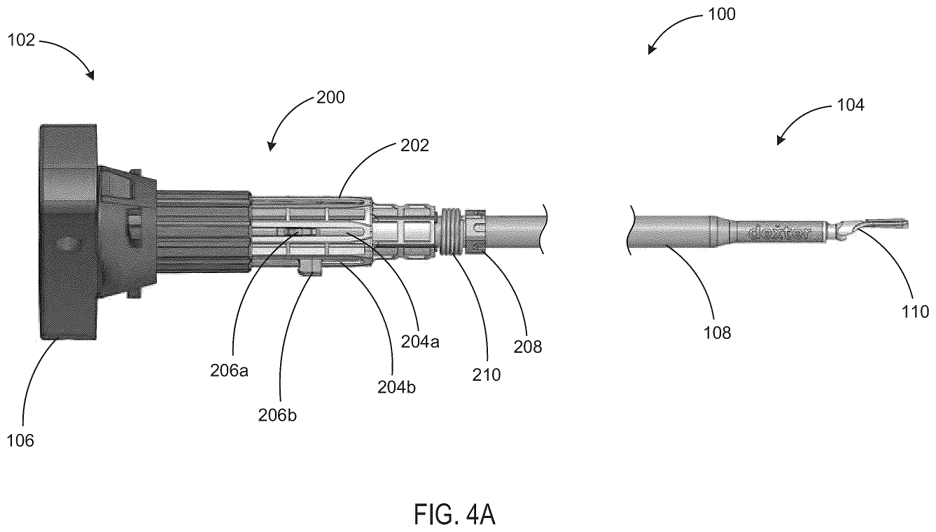

Referring now to FIGS. 4A and 4B, an exemplary surgical instrument is provided. Surgical instrument 100 may be interchangeable with various surgical platforms. For example, surgical instrument 100 may releasably engage with a surgical platform such as patient console 14 of surgical robot system 10, as described above. Additionally, surgical instrument 100 also may releasably engage with a surgical platform such a handheld surgical system, as described in further detail below. As shown in FIG. 4A, surgical instrument 100 may include surgical instrument interface 200 operatively coupled to end-effector 110 via instrument shaft 108. Accordingly, actuation of surgical instrument interface 200, e.g., via the engagers of patient hub 50, causes actuation of end-effector 110 via instrument shaft 108.

As shown in FIGS. 4A and 4B, surgical instrument interface 200 includes housing 202 having one or more longitudinal openings, and preferably, housing 202 has two longitudinal openings 204a, 204b. In addition, surgical instrument interface 200 includes barrel 212 rotatably disposed within and concentric with housing 202. Barrel 212 is coupled to instrument shaft 108. In some embodiments, barrel 212 may be coupled to instrument shaft 108 via torsion spring 210 and fixation ring 208. For example, proximal end 209 of spring 210 may be fixedly coupled to barrel 212, and distal end 211 of spring 210 may be fixedly coupled to fixation ring 208, which may be fixedly coupled to instrument shaft 108. Spring 210 may be pre-loaded with a predetermined torque by rotating fixation spring 208 relative to barrel 212, prior to fixing fixation ring 208 to instrument shaft 108. When the predetermined torque is achieved, fixation ring 208 may then be fixed to instrument shaft 108. The predetermined torque determines the maximum torque that may be transmitted between barrel 212 and instrument shaft 108 before torsional compression of spring 210, as described in further detail below.

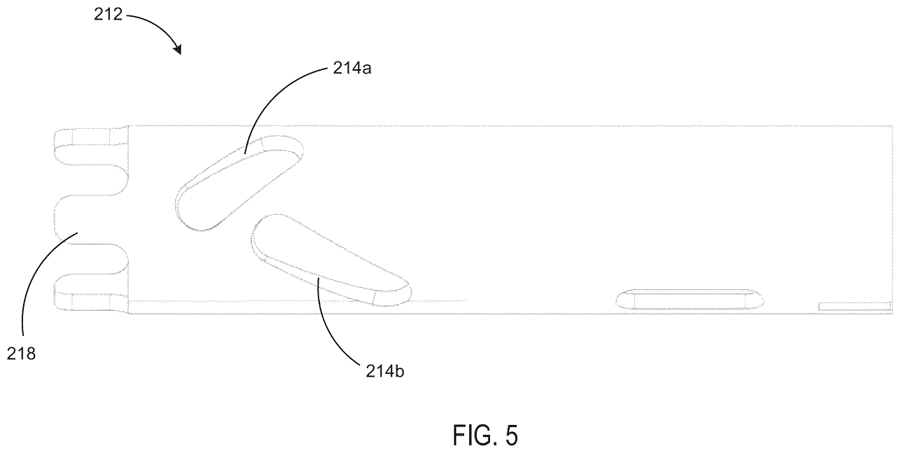

Moreover, as shown in FIGS. 4A and 4B, surgical instrument interface 200 includes one or more actuators, and preferably at least a pair of actuators 206a, 206b, slidably disposed within longitudinal openings 204a, 204b, respectively. Actuators 206a, 206b are sized and shaped to engage with the engagers of patient hub 50, e.g., via sterile shield 80. Accordingly, translational movement of receptacle 70a, and accordingly the engager attached thereto, in a first direction causes a corresponding translation movement of actuator 206a in the first direction within longitudinal opening 204a, and translational movement of receptacle 70b, and accordingly the engager attached thereto, in a second direction opposite to the first direction causes a corresponding translation movement of actuator 206b in the second direction within longitudinal opening 204b. In addition, each actuator 206a, 206b has engagement portion 207a, 207b (as shown in FIGS. 6A and 6B), respectively, extending radially inwardly toward the longitudinal axis of surgical instrument interface 200 through longitudinal openings 204a, 204b, respectively. Although FIGS. 4A and 4B show surgical instrument interface 200 having two actuators for actuating end-effector 110 in a degree-of-freedom, e.g., open/close, as will be understood by a person having ordinary skill in the art, in one embodiment, surgical instrument interface 200 may include only one actuator slidably disposed within a single longitudinal opening of housing 200 for actuating end-effector 110 in the open/close degree-of-freedom.

Engagement portions 207a, 207b slidably engage with a grooved opening of barrel 212, such that translation movement of actuator 206a, 206b causes barrel 212 to rotate along the grooved opening, as described in further detail below. Accordingly, when the torque generated by rotation of barrel 212 between barrel 212 and instrument shaft 108 is less than the predetermined torque of spring 210, rotation of barrel 212 causes instrument shaft 108 to rotate via spring 210. When the torque generated by rotation of barrel 212 between barrel 212 and instrument shaft 108 is greater than the predetermined torque of spring 210, rotation of barrel 212 causes spring 210 to compress torsionally, such that rotation torque of barrel 212 limits the torque transmitted to instrument shaft 108 to the torsion torque of spring 210, and accordingly, instrument shaft 108 may not rotate as barrel 212 rotates.

In addition, surgical instrument interface 200 may include identifier element 106 for permitting the control system of surgical robot system 10 to identify surgical instrument 100, as described in International Patent Appl. Pub. No. WO 2020/141487 to Chassot. For example, identifier element 106 may be an RFID token integrated with the instrument, which contains information on the kinematic configuration of surgical instrument 100.

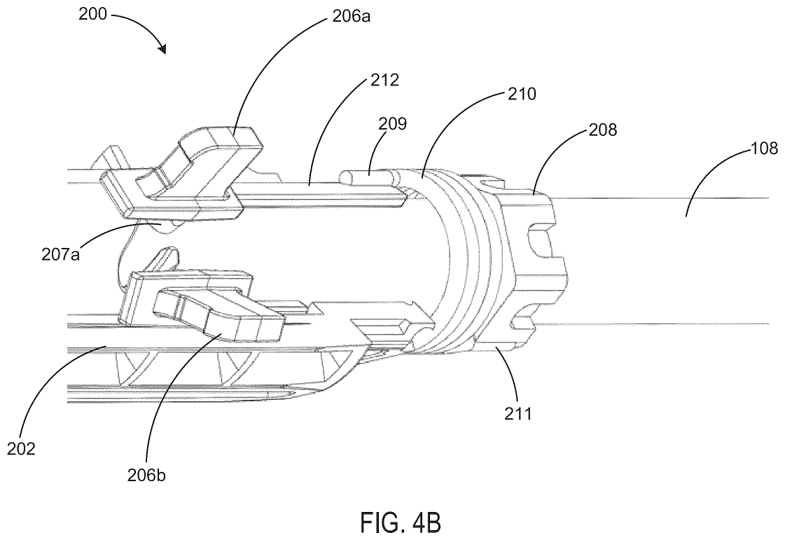

Referring now to FIG. 5, an exemplary barrel is provided. Barrel 212 is rotatably disposed within and concentric with housing 202, and coupled to instrument shaft 108. For example, barrel 212 may be coupled to instrument shaft 108 via spring 110, as described above. As shown in FIG. 5, barrel 212 includes one or more grooved openings 214a, 214b extending circumferentially about at least a portion of barrel 212. Grooved openings 214a, 214b may be cut into barrel 212 and sized and shaped to slidably receive actuators 206a, 206b therethrough. Moreover, grooved openings 214a, 214b may be oppositely angled such that translational movement of actuators 206a, 206b within longitudinal openings 204a, 204b in equal and opposite directions causes barrel 212 to rotate along grooved openings 214a, 214b. In some embodiments, grooved openings 214a, 214b may be a single opening within barrel 212 having the oppositely angled geometries to permit equal and opposite translational movement of actuators 206a, 206b along the grooved opening. As further shown in FIG. 5, the proximal end of barrel 212 may include cam stop 218 for rotatably engaging with a cam ring fixed to instrument shaft 108 to facilitate rotation of instrument shaft 108, as described in further detail below. Cam stop 218 may include a plurality of fingers spatially and circumferentially disposed about the proximal end of barrel 212, and extending proximally from the proximal end of barrel 212.

Referring now to FIGS. 6A and 6B, exemplary actuators of the surgical instrument interface are provided. As shown in FIGS. 6A and 6B, engagement portions 207a, 207b of actuators 206a, 206b extend through longitudinal openings 204a, 204b of housing 202, respectively, and slidably engage with grooved openings 214a, 214b of barrel 212, respectively. FIG. 6A illustrates actuators 206a, 206b in a minimum position, such that actuator 206a is in its proximal-most position relative to longitudinal opening 204a and along grooved opening 214a, and actuator 206b is in its distal-most position relative to longitudinal opening 204b and along grooved opening 214b. In the minimum position, actuators 206a, 206b may be, e.g., 10 mm apart from each other, and spring 210 may be in its pre-loaded configuration. Moreover, as described in further detail below, in the minimum position, end-effector 110 may be in an open configuration.

Actuators 206a, 206b may be actuated, e.g., via the engagers of patient hub 50, such that actuator 206a translationally moves distally within longitudinal opening 204a and along grooved opening 214a, and actuator 206b translationally moves proximally within longitudinal opening 204b and along grooved opening 214b. FIG. 6B illustrates actuators 206a, 206b in a maximum position, such that actuator 206a is in its distal-most position relative to longitudinal opening 204a and along grooved opening 214a, and actuator 206b is in its proximal-most position relative to longitudinal opening 204b and along grooved opening 214b. Distal movement of actuator 206a within longitudinal opening 204a and proximal movement of actuator 206b within longitudinal opening 204b causes barrel 212 to rotate about the longitudinal axis of surgical instrument 100 due to engagement of engagement portions 207a, 207b with grooved openings 214a, 214b. For example, in the maximum position, barrel 212 may be rotated, e.g., 70 degrees from the minimum position. The amount of rotation of instrument shaft 108 due the rotation of barrel 212 may be dependent on the amount of torque generated between barrel 212 and instrument shaft 108, as described in further detail below. Moreover, the maximum amount of rotation of instrument shaft 108 may be dependent on the length and angle of grooved openings 214a, 214b of barrel 212. As shown in FIG. 6B, actuators 206a, 206b may be aligned in the maximum position. Moreover, as described in further detail below, in the maximum position, end-effector 110 may be in a closed configuration.

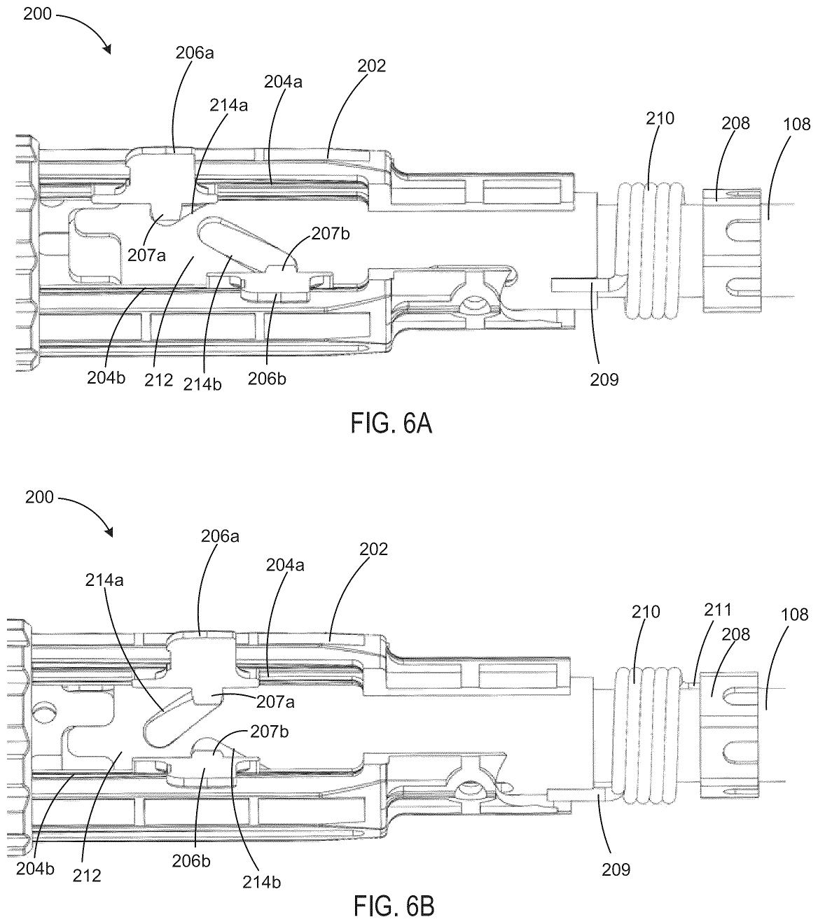

Referring now to FIGS. 7A and 7B, actuation of surgical instrument 100 when the torque generated between barrel 212 and instrument shaft 108 is less than the torque of spring 210 is described. FIG. 7A illustrates actuators 206a, 206b in the minimum position, e.g., actuator 206a is in its proximal-most position relative to grooved opening 214a and actuator 206b is in its distal-most position relative to grooved opening 214b. As shown in FIG. 7A, when going from the minimum position toward the maximum position, e.g., as actuator 206a moves distally in direction A and actuator 206b moves proximally in direction B, barrel 212 rotates in direction C due to engagement of actuators 206a, 206b with grooved openings 214a, 214b, respectively. In FIG. 7A, the actuation force of actuators 206a, 206b is such that the torque generated between barrel 212 and instrument shaft 108 is less than the predetermined torque of pre-loaded spring 210. Accordingly, rotation of barrel 212 in direction C causes rotation of instrument shaft 108 in direction D via spring 210 and fixation ring 208. As shown in FIG. 7A, cam ring 220 includes groove 222 sized and shaped to engage with cam stop 218, such that cam stop 218 may move circumferentially within groove 222. As both barrel 212 and instrument shaft 108 rotate due to actuation of actuators 206a, 206b when the torque generated between barrel 212 and instrument shaft 108 is less than the torque of spring 210, accordingly, both cam stop 218 of barrel 212 and cam ring 220 fixed to instrument shaft 108 also rotate.

As shown in FIG. 7B, when going from the maximum position toward the minimum position, e.g., as actuator 206a moves proximally in direction A and actuator 206b moves distally in direction B, barrel 212 rotates in direction C due to engagement of actuators 206a, 206b with grooved openings 214a, 214b, respectively. As barrel 212 rotates in direction C, cam stop 218 transmits torque to cam ring 220, e.g., within groove 222, to thereby cause cam ring 220, and accordingly instrument shaft 108, to rotate in direction D.

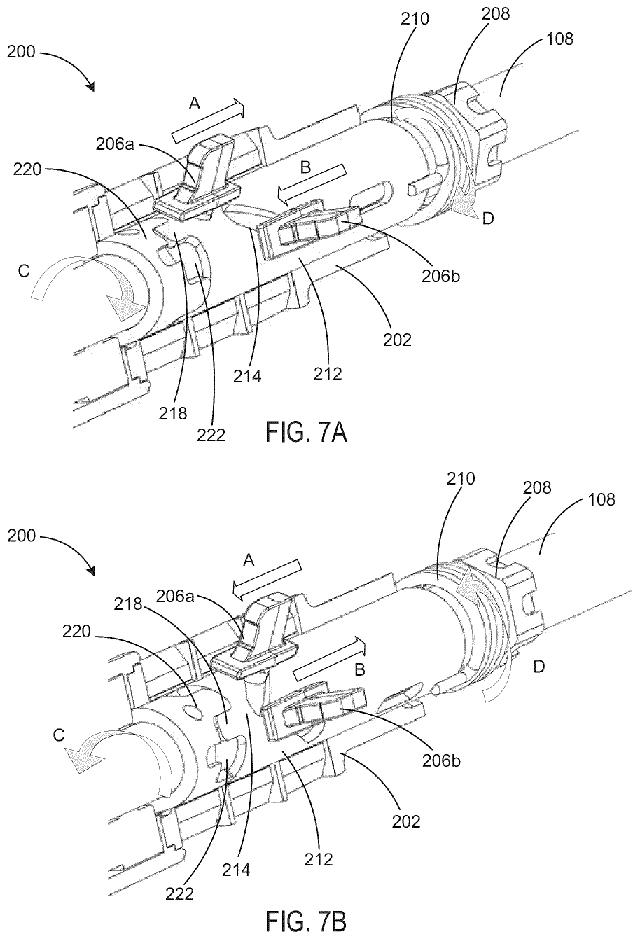

Referring now to FIGS. 8A and 8B, actuation of surgical instrument 100 when the torque generated between barrel 212 and instrument shaft 108 is greater than the torque of spring 210 is described. FIG. 8A illustrates actuators 206a, 206b in the minimum position, e.g., actuator 206a is in its proximal-most position relative to grooved opening 214a and actuator 206b is in its distal-most position relative to grooved opening 214b. As shown in FIG. 8A, when going from the minimum position toward the maximum position, e.g., as actuator 206a moves distally in direction A and actuator 206b moves proximally in direction B, barrel 212 rotates in direction C due to engagement of actuators 206a, 206b with grooved openings 214a, 214b, respectively. In FIG. 8A, the actuation force of actuators 206a, 206b is such that the torque generated between barrel 212 and instrument shaft 108 is greater than the predetermined torque of pre-loaded spring 210. Accordingly, spring 210 absorbs at least some of the torque generated between barrel 212 and instrument shaft 108, such that barrel 212 begins to rotate relative to instrument shaft 108 (instead of rotating synchronously with instrument shaft when the torque generated between barrel 212 and instrument shaft 108 is less than the predetermined torque of pre-loaded spring 210). Accordingly, spring 210 torsionally compresses, such that limited torque is transmitted from barrel 212 to instrument shaft 108 via spring 210. Accordingly, the maximum torque that the mechanism is able to transmit to instrument shaft 108 is equal to the sum of the preloaded predetermined torque of spring 210 and the additional rotation torque of the barrel multiplied by the stiffness of spring 210.

As shown in FIG. 8B, when actuators 206a, 206b are in the maximum position, and spring 210 is in a torsionally compressed configuration because the torque generated between barrel 212 and instrument shaft 108 due to actuation of actuators 206a, 206b is greater than the torque of spring 210, cam stop 218 is circumferentially moved relative to cam ring 220 within groove 218. This is because barrel 212 rotated, while rotation of instrument shaft 108 was limited. As further shown in FIG. 8B, when going from the maximum position toward the minimum position, e.g., as actuator 206a moves proximally in direction A and actuator 206b moves distally in direction B, barrel 212 rotates in direction C due to engagement of actuators 206a, 206b with grooved openings 214a, 214b, respectively. The torque generated from rotation of barrel 212 is first absorbed by spring 210, such that spring 210 torsionally releases its torque, until cam stop 218 engages with cam ring 220 within groove 222. Upon engagement of cam stop 218 with cam ring 220, further rotation of barrel 212 will cause torque to be transmitted from cam stop 218 to cam ring 220, to thereby cause corresponding rotation of instrument shaft 108.

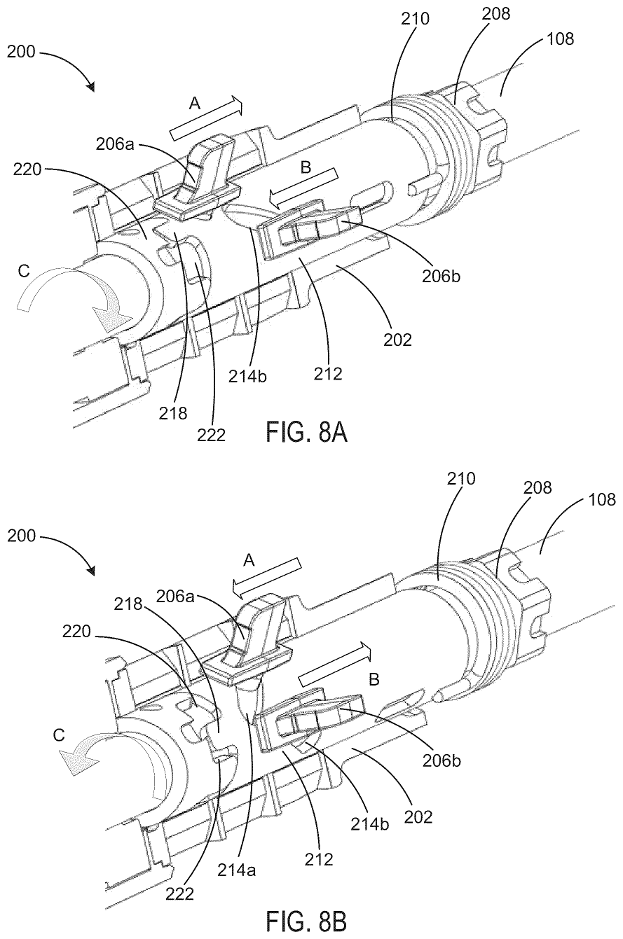

FIG. 9 is a graph illustrating the transmission of torque from barrel 212 to instrument shaft 108 relative to the force applied to actuators 206a, 206b. As shown in FIG. 9, when the rotation barrel torque generated via force applied to actuators 206a, 206b is less than the predetermined torque of spring 210, the amount of torque transmitted from barrel 212 to instrument shaft 108 is equal to the rotation barrel torque, such that instrument shaft 108 rotates synchronously with barrel 212. Moreover, when the rotation barrel torque generated via force applied to actuators 206a, 206b is greater than the predetermined torque of spring 210, spring 210 limits the torque transmitted from barrel 212 to instrument shaft 108 as more force is applied to actuators 206a, 206b. The slope of the first portion (a) depends on the shape of the cam (e.g., grooved openings 214a, 214b) and on the yield of the transmission. The slope of the second portion (b) depends exclusively on the spring rate.

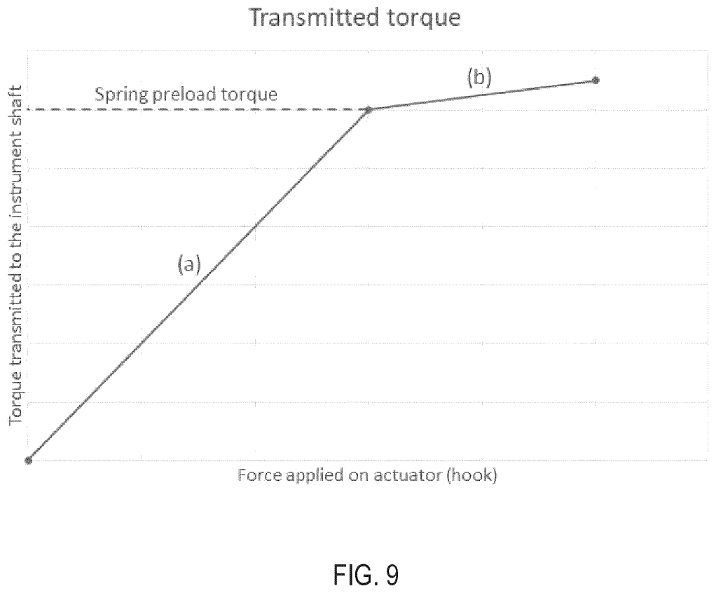

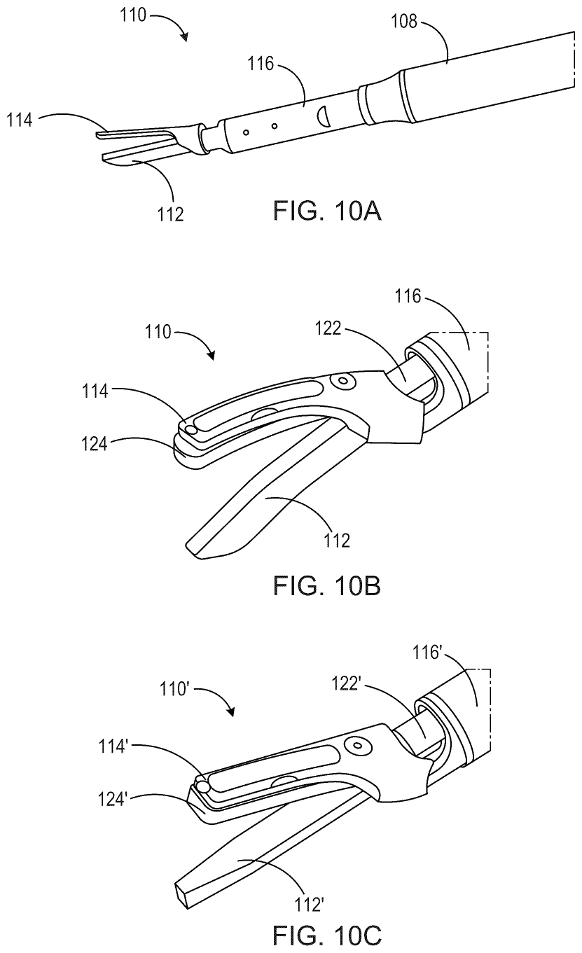

Referring now to FIGS. 10A and 10B, an exemplary end-effector is provided. End-effector 110 may be coupled to instrument shaft 108 via extension portion 116, which is fixed to instrument shaft 108. Alternatively, end-effector 110 may be directly coupled to instrument shaft 108 without extension portion 116. As shown in FIG. 10B, end-effector 110 may include a pair of jaws, e.g., jaws 112, 114. Jaw 112 may be fixedly coupled to rod 122, which remains stationary relative to housing 202 of surgical instrument interface 200 during operation of surgical instrument 100. Accordingly, rod 122 and jaw 112 may remain stationary as instrument shaft 108 rotates as described above.

Moreover, end-effector 110 may be configured to emit ultrasound energy to facilitate cutting and coagulation by the surgeon using surgical instrument 100. For example, end-effector 110 may be a Lotus ultrasonic scalpel (made available by BOWA-electronic GmbH & Co. KG, Gomaringen, Germany). Accordingly, ultrasonic energy may be transmitted via jaw 112 during actuation of surgical instrument 110. As shown in FIG. 10B, jaw 112 may have a narrow blade with focusing grooves for accurate dissection. Moreover, jaw 114 may include electrically insulated portion 124, which contacts jaw 112 in the closed position, thereby facilitating cutting and coagulation by end-effector 110. Alternatively, as shown in FIG. 10C, jaw 112′ may have a larger contact surface, thereby providing a stronger hemostatic effect.

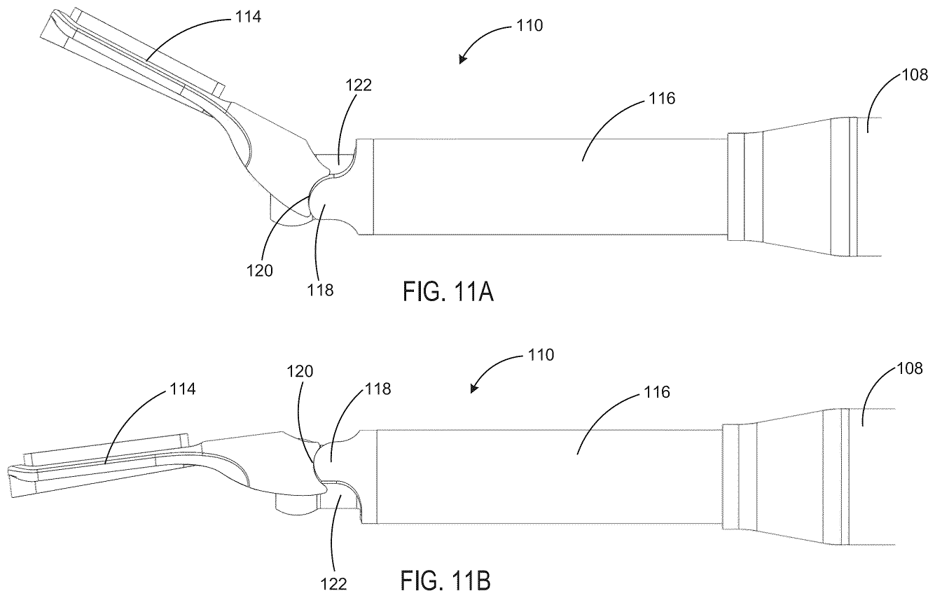

Referring now to FIGS. 11A and 11B, actuation of the end-effector of surgical instrument 100 is described. End-effector 110 may be operatively coupled to extension portion 116 of instrument shaft 108 via a cam mechanism, as described in U.S. 2005/0216045 to Young, the entire contents of which are incorporated herein by reference. Specifically, as shown in FIG. 11A, extension portion 116 may include cam 118, and jaw 114 may include follower 120 having a geometry sized and shaped to engage with cam 118. Jaw 112 is omitted for brevity. As instrument shaft 108, and accordingly extension portion 116 and cam 118 rotates, jaw 114 transitions between an open configuration in the minimum position and a closed configuration in the maximum position due to the engagement of cam 118 and follower 120. For example, as shown in FIG. 11A, in the minimum position, jaw 114 is in its open configuration.

As shown in FIG. 11B, when instrument shaft 108, and accordingly extension portion 116 and cam 118 rotates from the minimum position to the maximum position, the rotation of cam 118 causes jaw 114 to transition from the open configuration to the closed configuration as follower 120 pivots about cam 118. Accordingly, cables are not required to be routed through instrument shaft 108 of surgical instrument 100 in order to actuate end-effector 110. Jaws 112 and 114 may dissect tissue as jaw 114 transitions from the open configuration to the closed configuration. Surgical instrument 100 further may be actuated to emit ultrasonic energy to facilitate cutting and coagulation of the tissue during operation of surgical instrument 100.

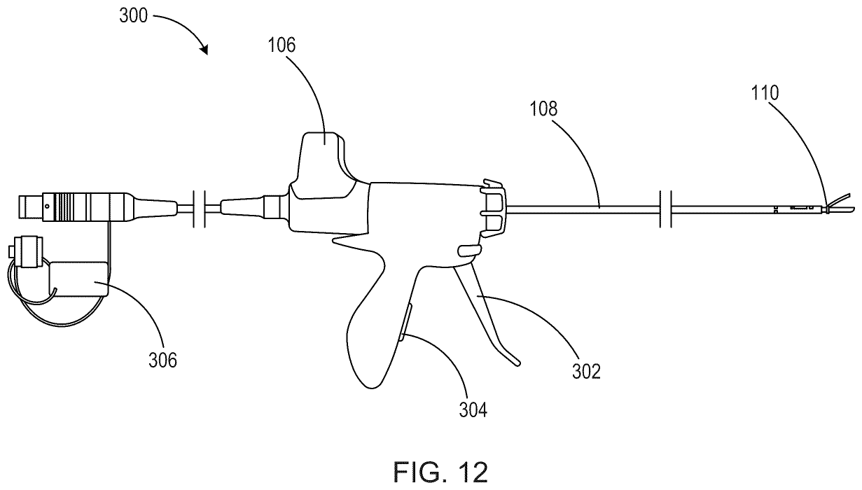

Referring now to FIG. 12, surgical instrument may be releasably engaged with an exemplary handheld surgical system. Handheld surgical system 300 may be a Lotus system (made available by BOWA-electronic Gmbh & Co. KG, Gomaringen, Germany). As shown in FIG. 12, system 300 may include trigger 302, power actuator 304, and transducer 306. Trigger 302 may be actuated by the surgeon to cause translational movement of engagers of system 300, which are engaged with actuators 206a, 206b when surgical instrument 100 is coupled to system 300. Accordingly, translational movement of the engagers of system 300 will cause translational movement of actuators 206a, 206b, which will cause rotation of instrument shaft 108 if the torque generated between barrel 212 and instrument shaft 108 is less than the predetermined torque of spring 210, or will not cause rotation of instrument shaft due to compression of spring 210 if the torque generated between barrel 212 and instrument shaft 108 is greater than the predetermined torque of spring 210, as described above. In addition, power actuator 304 of system 300 may be actuated to cause end-effector 110 to emit ultrasonic energy during operation of system 300. Transducer 306 provides power for actuating end-effector 110 via power actuator 304, and may be releasably coupled to system 300. Moreover, transducer 306 may be reusable.

While various illustrative embodiments of the invention are described above, it will be apparent to one skilled in the art that various changes and modifications may be made therein without departing from the invention. The appended claims are intended to cover all such changes and modifications that fall within the true scope of the invention.

1. A surgical instrument interface operatively coupled to an instrument shaft having an end-effector, the surgical instrument interface comprising:
a housing comprising a first longitudinal opening and a second longitudinal opening;
a barrel rotatably disposed within the housing, the barrel comprising a first grooved opening and a second grooved opening each extending along at least a portion of the barrel, the first and second grooved openings being oppositely angled with respect to one another; and
a first actuator slidably disposed within the first longitudinal opening and engaged with the first grooved opening and a second actuator slidably disposed within the second longitudinal opening and engaged with the second grooved opening, such that translational movement of the first and second actuators along the first and second longitudinal openings, respectively, causes the barrel to rotate along the first and second grooved openings to thereby rotate the instrument shaft.

2. The surgical instrument interface of claim 1, further comprising:
a spring having a proximal end coupled to the barrel and a distal end coupled to the instrument shaft, the spring configured to be pre-loaded with a predetermined torque,
wherein translational movement of the first and second actuator along the respective first and second longitudinal opening causes the barrel to rotate along the first and second grooved openings to thereby rotate the instrument shaft via the spring.

3. The surgical instrument interface of claim 2, wherein rotation of the barrel causes the instrument shaft to rotate when a torque generated by rotation of the barrel between the barrel and the instrument shaft is less than the predetermined torque of the spring.

4. The surgical instrument interface of claim 3, wherein, when the torque generated by rotation of the barrel between the barrel and the instrument shaft is less than the predetermined torque of the spring, the spring is configured to transmit the torque to the instrument shaft to rotate the instrument shaft in a first direction.

5. The surgical instrument interface of claim 4, wherein the barrel comprises a cam stop configured to engage with a cam ring fixed to the instrument shaft, and wherein, when the torque generated by rotation of the barrel between the barrel and the instrument shaft is less than the predetermined torque of the spring, the cam stop is configured to transmit the torque to the instrument shaft via the cam ring to rotate the instrument shaft in a second direction opposite to the first direction.

6. The surgical instrument interface of claim 5, wherein the cam stop extends proximally from a proximal end of the barrel, and wherein the cam ring comprises a distal opening sized and shaped to receive the cam stop and to permit the cam stop to circumferentially move within the distal opening.

7. The surgical instrument interface of claim 2, wherein, when a torque generated by rotation of the barrel between the barrel and the instrument shaft is greater than the predetermined torque of the spring, rotation of the barrel in a first direction causes the spring to compress torsionally such that rotation torque of the barrel in the first direction limits the torque transmitted to the instrument shaft to the torsion torque of the spring.

8. The surgical instrument interface of claim 7, wherein, rotation of the barrel in a second direction opposite to the first direction causes the spring to absorb movement of the barrel until the barrel is able to transmit torque to the instrument shaft.

9. The surgical instrument interface of claim 2, wherein the distal end of the spring is coupled to the instrument shaft via a fixation ring, the fixation ring configured to be actuated to pre-load the spring with the predetermined torque.

10. (canceled)

11. The surgical instrument interface of claim 1, wherein the first and second grooved openings, by being oppositely angled, are configured such that translational movement of the first and second actuators in opposite directions along the respective first and second longitudinal openings rotates the barrel.

12. (canceled)

13. The surgical instrument interface of claim 1, wherein the end-effector is configured to emit ultrasonic energy.

14. The surgical instrument interface of claim 1, wherein the end-effector comprises a pair of jaws.

15. The surgical instrument interface of claim 13, wherein rotation of the instrument shaft causes a first jaw of the pair of jaws to move relative to a second jaw of the pair of jaws of the end-effector to thereby cause the end-effector to open and close.

16. The surgical instrument interface of claim 14, wherein the first jaw is operatively coupled to the instrument shaft via a cam mechanism such that rotation of the instrument shaft causes the end-effector to open and close.

17. A surgical instrument configured to be attached to a surgical platform, the surgical instrument comprising:
the surgical instrument interface of claim 1;
an instrument shaft operatively coupled to the surgical instrument interface; and
an end-effector disposed at a distal end of the instrument shaft.

18. A surgical robot system configured to be releasably coupled to the surgical instrument of claim 17, the system further comprising a surgical platform having a patient console, the patient console configured to releasably engage with the actuator of the surgical instrument interface.

19.-24. (canceled)

25. The surgical instrument interface of claim 1, wherein the first and second grooved openings collectively form a single opening in the barrel.
